(19) United States
(12) Patent Application Publication
(10) Pub. No.: US 2013/0324945 A1
(43) Pub. Date: Dec. 5, 2013

(54) PERCUTANEOUS CONNECTION ASSEMBLY FOR ACTIVE MEDICAL DEVICES

(71) Applicant: Pierre Sabin, Paris (FR)

(72) Inventor: Pierre Sabin, Paris (FR)

(21) Appl. No.: 13/905,855

(22) Filed: May 30, 2013

Related U.S. Application Data
(60) Provisional application No. 61/653,304, filed on May 30, 2012.

(51) Int. Cl. A61N 1/05; A61N 5/06; A61M 5/00

(52) U.S. Cl. CPC A61N 1/05; A61M 5/00; A61N 5/0613
USPC 604/257; 607/116; 607/88

(57) ABSTRACT

The invention concerns a novel medical system intended to be fixed in a surgically prepared osseous site of a patient and that connects external components to active entities located inside the body of the patient.

External components and active entities are designed to store, emit and/or transfer a specific flow, such as a flow of electricity, data, fluids, solids or light.

The external components can be housed in an external housing and bound to active entities being located within the body of the patient through connection elements located within a percutaneous abutment, an implant and possible additional implanted means of connections and/or stimulations.

### CROSS-REFERENCE TO RELATED APPLICATIONS

This application claims the benefit of the filing date of U.S. Provisional Patent Application No. 61/653,304 filed May 30, 2012, the disclosure of which is hereby incorporated herein by reference.

### FIELD OF INVENTION

The present invention relates to the implantation of medical devices in the body of an animal, in particular in the human body, especially the implantation of active medical devices, more particularly to set up an electric, liquid or optic connection between one or several external component to said body and one or several active elements implanted in said body.

### BACKGROUND OF THE INVENTION

Active medical devices have expanded in multiple therapies spanning from cardiology to neurology and including urology as well as gastroenterology. The most common type of device is a fully-implanted electrical stimulator reckoning on an internal battery and embedded electronic components. This architecture is faced with two main drawbacks: battery life limitations and the need to use expensive embedded components.

As the battery gets exhausted the patient need to undertake surgery in order to replace the whole device. This device replacement procedure adds additional procedure costs and device costs to the therapy, increases the risks of the therapy and lowers the quality of life of the patients. Despite recent progress in battery cells and the advent of wirelessly rechargeable system that have extended the active medical device battery lives, batteries remain a limitation to active medical device therapies.

Components used—especially electronics that generate the electrical stimulation—need to compel with the demands of long term implantation. Therefore, high-end components are used and push the devices prices up.

The two drawbacks described above limit the large scale adoption of these electrical based therapies.

Permanent percutaneous devices have gained momentum in Otorhinolaryngology (e.g. bone anchored hearing assistance) and Oral Maxillofacial Surgery (e.g. facial reconstruction with bone anchored prosthetics supports). However, such percutaneous devices cannot transfer electricity, liquids or optics to the human body.

### SUMMARY OF THE INVENTION

The present invention relates to a novel percutaneous device assembly designed for active medical devices.

More precisely, it relates to a percutaneous connection assembly intended to be fixed by osseointegration in an osseous structure of a patient for transfer of a flow—preferably of electricity, light, data, or matter—between the interior and exterior of the body of the patient, wherein the assembly comprises:
An implant device having:
   a percutaneous socket having a first end comprising a percutaneous abutment (120) and a second end opposite to the first end;
   an elongated extension member (130) intended to be inserted within a hole created into the osseous structure, said extension member (130) having a first end coupled to the second end of the socket, and a second end opposite to the first end, the coupling of the extension member (130) relative to the percutaneous socket being designed for angular shifting of the first end of the percutaneous socket relative to the second end of the extension member (130); and
   connection elements running through the device from the first end of the percutaneous socket to the second end of the extension member (130), said connection elements comprising at least a first connector arranged within the percutaneous abutment (120);
An external device for emission and/or reception of the flow toward and/or from the second end of the extension member (130), said external device including a housing (110) with coupling elements for direct coupling of the housing onto the percutaneous abutment (120) external to the body of the patient.

Preferable but non limiting aspects of such assembly, taken alone or in combination, are the following:
the external device is configured to convey an electrical signal, the housing of the external device containing one or several of elements among batteries, electronics, cables, connectors, circuitry, coils and any other elements for emission, reception, transfer, storage and/or processing of electrical signals.
the external device is configured to convey liquids and/or solids, the housing of the external device containing one or several of elements among pipes, tanks, valves, and/or pumps, and any other elements for emission, reception, transfer, storage and/or treatment liquids and/or solids.
the external device is configured to convey light, the external housing containing one or several of elements among laser and associated electronics, optical fibers, sensors and associated electronics, LED, mirrors, and any other optical elements for emission, reception, transfer, and/or storage of light.

There is also proposed a percutaneous connection device intended to be fixed by osseointegration in an osseous structure of a patient for transfer of a flow—preferably of electricity, light, data, or matter—between inside the body of the patient and an entity external to said body, wherein the device comprises:
a percutaneous socket having a first end comprising a percutaneous abutment (120) and a second end opposite to the first end;
an elongated extension member (130) intended to be inserted within a hole created into the osseous structure, said extension member (130) having a first end coupled to the second end of the socket, and a second end opposite to the first end, the coupling of the extension member (13) relative to the percutaneous socket being designed for angular shifting of the first end of the percutaneous socket relative to the second end of the extension member (130);
connection elements running through the device from the first end of the percutaneous socket to the second end of the extension member (130), said connection elements comprising at least a first connector arranged within the percutaneous abutment (120) on one side of the osseous structure; and
a connection housing coupled at the second end of the elongated extension member (130), said connection housing being designed for bringing a second connector of the connection elements on the other side of the osseous structure.

Preferable but non limiting aspects of such device, taken alone or in combination, are the following:
the connection housing is directly connected to the elongated extension member (130), said connection housing being designed to enclose several components therein.
the connection housing comprises at least a capsule coupled to the elongated extension member (130) with a flexible connecting member, said capsule having a design enabling a passage through the osseous structure.
the device comprises an external device for emission and/or reception of the flow toward and/or from the second end of the extension member (130), said external device including a housing (110) with coupling elements for direct coupling of the housing onto the percutaneous abutment (120) external to the body of the patient.

### BRIEF DESCRIPTION OF THE DRAWINGS

Other advantages and characteristics of the invention will emerge from the detailed description of non-limiting embodiments, and from the attached drawings, wherein:

FIG. 1A illustrates a side view of the percutaneous system implanted and surrounding body tissues

FIG. 1B illustrates a side view of a percutaneous platform associated with a stimulating electrode and surrounding body tissues

FIG. 1C illustrates a side view of a percutaneous platform associated with a housing interface assembly and surrounding body tissues

FIG. 1D illustrates a side view of a percutaneous platform associated with a cable assembly and surrounding body tissues

FIG. 2A illustrates a first embodiment of anchoring means at one of the implant extremity.

FIG. 2B illustrates a first embodiment of anchoring means at one of the implant extremity.

### DETAILED DESCRIPTION

The invention concerns a novel medical system intended to be fixed in a surgically prepared osseous site of a patient and that connects external components to active entities located inside the body of the patient.

External components and active entities are designed to store, emit and/or transfer a specific flow, such as a flow of electricity, data, fluids, solids or light.

The external components can be housed in an external housing (110) and bound to active entities being located within the body of the patient through connection elements located within a percutaneous abutment (120), an implant (130) and possible additional implanted means of connections and/or stimulations.

The design of the housing (110) depends on its functions, and all the components it houses may also be changed depending on the function to be performed.

The housing (110) is coupled to the percutaneous abutment (120) at the exterior of the body of the patient. The coupling of such external housing (110) with regard to the percutaneous abutment (120) may be removable, but the housing (110) can also be securely fastened to the percutaneous abutment (120). The external housing can for example be connected remotely (contactless), mechanically (including notably screw systems and clip-in systems), magnetically, and/or chemically to the abutment (120).

Three main set-ups can be considered for the external housing (110).

When the purpose of the external housing is to convey electricity, the external housing (110) may for example contain one or several of the following elements intended for communication emission and/or transfer; and/or energy emission, transfer and/or storage:
battery,
electronics,
cable,
connector,
circuitry,
coils.

When the purpose of the external housing is to convey liquids and/or solids; the external housing (110) may for example contain pipes, tanks, valves, and/or pumps, intended to transfer, store and/or treat liquids and/or solids.

For any optical purposes, the external housing (110) may contain laser and associated electronics, optical fibers, sensors and associated electronics, LED, mirrors, or other optical systems intended to emit, receive, transfer and/or store light.

The abutment (120) and the implant (130) preferably contain means of connection. For electric transfer, connectors, wires or circuitry are considered. Alternatively it can contain means to transfer liquids (e.g. tubes) or light (e.g. optical fibers).

The percutaneous abutment (120) can be monoboc with the external housing (110) and/or monobloc with the endosseous implant (130).

The implant (130) is preferably in titanium to favour osseointegration. If designed for short term implantation, alternative materials can be considered such as Polyether ether ketone (PEEK).

The implant (130) can contain one or several of the following elements:

one or several means of electrical connections; electronics circuitry;
one or several means of optical connections;
one or several pipes, tanks and/or pumps, intended to transfer, store and/or treat liquids and/or solids,

Several implant embodiments are considered.

In a first embodiment the implant consists in a base and a tubular extension.

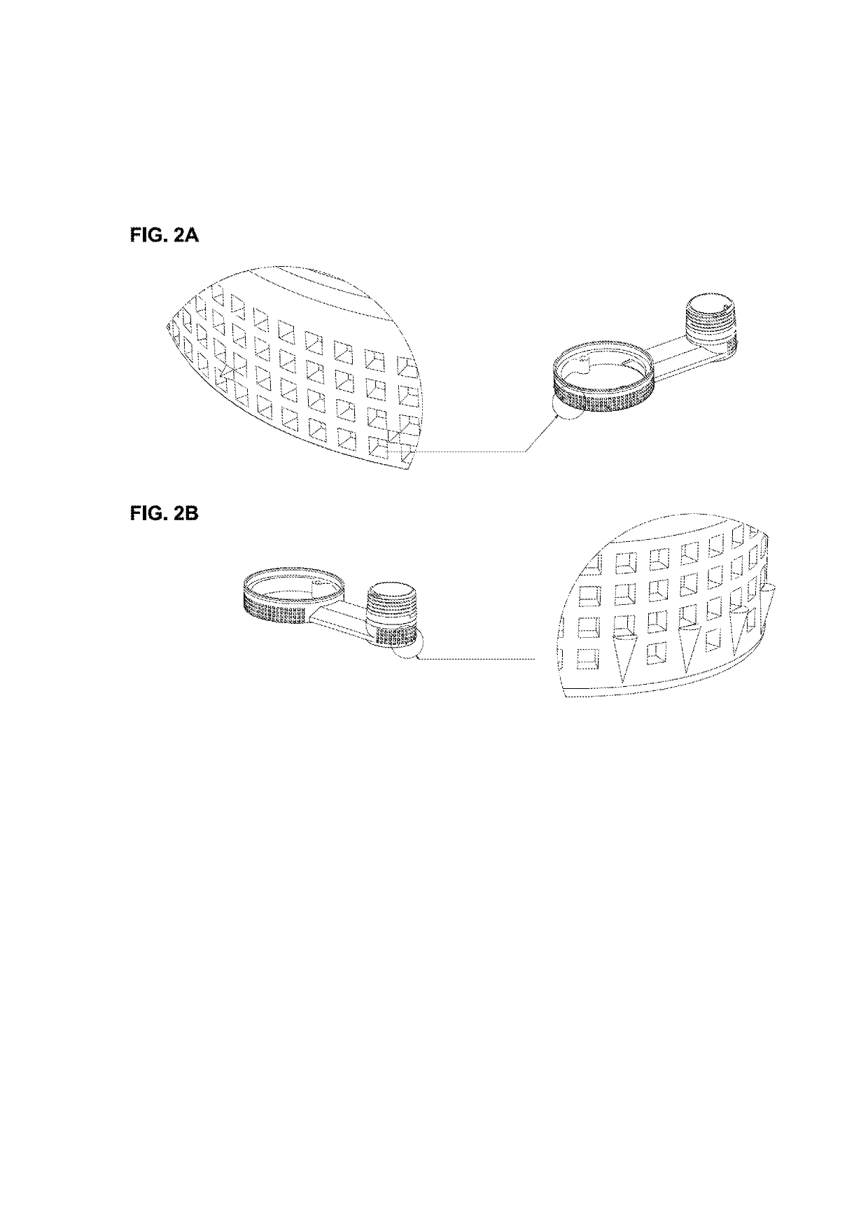

In order to secure bone anchorage the implant contains one or several anchoring means, that are designed for osseointegration of the implant in the osseous structure. At least three anchoring systems are considered:
Cams; one or several cams are extending from the implant. The cams are activated through strips so that the external surface of the cams protrudes into the osseous structure wall maintaining therein the implant in position.
Screws; plates are extending from the implant or alternatively independent plates are placed over the implant by the surgeon during the surgery. The plate has at least one through orifice intended to receive an osteosynthesis screw which are used for anchoring the implant in the osseous structure.
Hooking system; the surface of implant is characterized by projections protruding relative to the surface of the osseous structure so that the implant can be impacted into the osseous structure (FIGS. 2A and 2B).

Additionally, the outer surfaces of the implant may be provided with some cavities in order to optimise bone generation into the cavities, and thus guarantee long term anchorage.

In a second embodiment the base and one or several tubular extension are not monobloc.

In this second embodiment, at least two anchoring means can be considered:
A threaded system, wherein the base is threaded and preferably spherical shaped so that it can be screwed into the osseous structure.
A hooking system, wherein the surface of the base is characterized by projections protruding relative to the surface of the osseous structure so that the implant can be impacted into the osseous structure.

The percutaneous system is designed to maintain mechanically the external elements and offer a percutaneous connection that is resistant from pathogens penetration. Therefore the implant geometry allow to anchor the implant in the osseous structure (1), to reduce the dermis (3) around the percutaneous passage so that the epithelium (4) can be sutured on the periosteum (2) forming an epithelial seal against pathogens. To lower the risk of adverse events the implant (130) should preferably implanted in the calvaria.

One preferred example on the percutaneous connection assembly that can be used is described in the PCT application published on Aug. 2, 2012 under the reference WO 2012/101267, which is herewith incorporated by reference.

It can also be used a percutaneous connection device, preferably intended to transfer energy or matter, to be fixed in an osseous structure of a patient to connect an internal entity located inside the body of the patient to an entity external to said body, wherein the device comprises:
a percutaneous socket having a first end comprising a percutaneous abutment and a second end opposite to the first end;
an elongated extension member intended to be inserted within a hole created into the osseous structure, said extension member having a first end comprising means to be removably coupled to the second end of the socket, and a second end opposite to the first end, the removable coupling of the extension member relative to the percutaneous socket being designed for angular shifting of the first end of the percutaneous socket relative to the second end of the extension member;
anchoring means provided for anchoring the device to the osseous structure by osseointegration; and
connection means running through the device from the first end of the percutaneous socket to the second end of the extension member, said connection means comprising at least a first connector arranged within the percutaneous abutment.

Preferable but non limiting aspects of such device, taken alone or in combination, are the following:
the extension member is removably fastened to the percutaneous socket, and the anchoring means are arranged at the second end of the percutaneous socket, said second end of the percutaneous socket being designed for osseous burial in the osseous structure so that the percutaneous abutment protrudes relative to the surface of the osseous structure.
the percutaneous socket is removably fastened to the extension member, and the anchoring means are arranged at the first end of the extension member, close to the removable coupling of the extension member relative to the percutaneous socket.
the extension member and the percutaneous socket are removably coupled via an anchoring base comprising the anchoring means, said anchoring base being designed for osseous burial in the osseous structure and comprising first coupling means for removable fastening of the percutaneous abutment intended to protrude relative to the surface of the osseous structure, and second coupling means for removable fastening of the extension member intended to be fully buried in the osseous structure.

Alternatively, it can be used a percutaneous connection device, preferably intended to transfer energy or matter, to be fixed in an osseous structure of a patient to connect an internal entity located inside the body of the patient to an entity external to said body, wherein the device comprises:
an implant having a cylindrical or truncated-conical shape, said implant forming an anchoring base with anchoring means for anchoring of the device in the osseous structure, and said implant having at least one lateral hole provided in a lateral wall of the implant;
a percutaneous abutment having a shape to be at least partially inserted in the implant, preferably in a removable manner;
an elongated extension member intended to be inserted within a hole created into the osseous structure, said extension member having a first end comprising means to be removably inserted within the lateral hole of the implant, and a second end opposite to the first end; and
connection means running through the device from the percutaneous abutment to the second end of extension member, said connection means comprising at least a first connector arranged within the percutaneous abutment.

Preferable but non limiting aspects of such device, taken alone or in combination, are the following:
the implant comprises a threaded portion for implantation into the osseous structure, and a ring portion at one end of the implant for tightening and adjusting the position of the implant into the osseous structure.
the ring portion has a polygonal external shape, preferably an octagonal of hexagonal external shape.
the ring portion comprises at least one lateral flat portion.
the anchoring means comprises at least one anchoring element arranged so as to be able to protrude relative to the surface the device in order to mesh with a lateral wall of a cavity of the osseous structure.
the anchoring means comprises a threaded surface, said threaded surface easing primary anchoring of the device in the osseous structure.
the anchoring element comprises projecting portions, said projecting portions being designed to penetrate the lateral wall of the cavity in a depth between 20 micrometres and 2000 micrometres, and preferably in a depth of 400 micrometres.
the projecting portions have a geometric shape to provide a retention effect, said shape being preferably a symmetric shape chosen among a cone shape, a pyramid shape, and/or a polyhedron shape.
the percutaneous socket, the percutaneous abutment, the implant, and/or the extension member are made of titanium, polyether ether ketone, zirconia and/or any biocompatible material.
the implant is made of titanium using machining and/or additive manufacturing processes.
the implant and/or the extension member is coated with a coating for promoting osseointegration of the device into the osseous structure.
the percutaneous abutment can be connected mechanically, magnetically, and/or physically to one or multiple external parts.
the first connector is clipped within the percutaneous abutment with a non-return system.
the first connector is maintained in position within the percutaneous abutment with a maintaining element inserted within the percutaneous abutment.
the maintaining element is a ring screwed or pushed in the abutment, said ring preferably comprising a cutting on the inside in order to place an O-ring to maintain the first connector in compression.
the extension member has a tubular lumen geometry, said tubular lumen geometry being chosen among parallelepipedal, regular polygonal, irregular polygonal circular, ovaloid, round or a combination thereof.
the extension member comprises a plurality of tubes, which might be arranged parallel to each other.
the first connector is connected to a subcutaneous connector by a ribbon cable made of biocompatible electrical wires, encapsulated with silicon or any other material that is both flexible and biocompatible.
the device comprises a subcutaneous connector intended to be connected to the internal entity, wherein said subcutaneous connector comprises a screw or pin system to lock and seal the subcutaneous connector.
the device comprises a subcutaneous connector intended to be connected to the internal entity, wherein said subcutaneous connector comprises at least one eyelet on each side, said eyelets being used to attach the implant to the bone with screw and/or suture the implant to the fascia.
the abutment is partly and/or totally cylindrical, triangular and/or polygonal.
the connection means comprises a second connector arranged at the opposite end of the connection means relative to the first connector, said second connector having a shape designed to pass through the percutaneous socket and the extension member.
the connection means are electrical connection means, and the first connector and/or the second connector are jack connectors, preferably having a cross-section being circular or in cross arrangement.

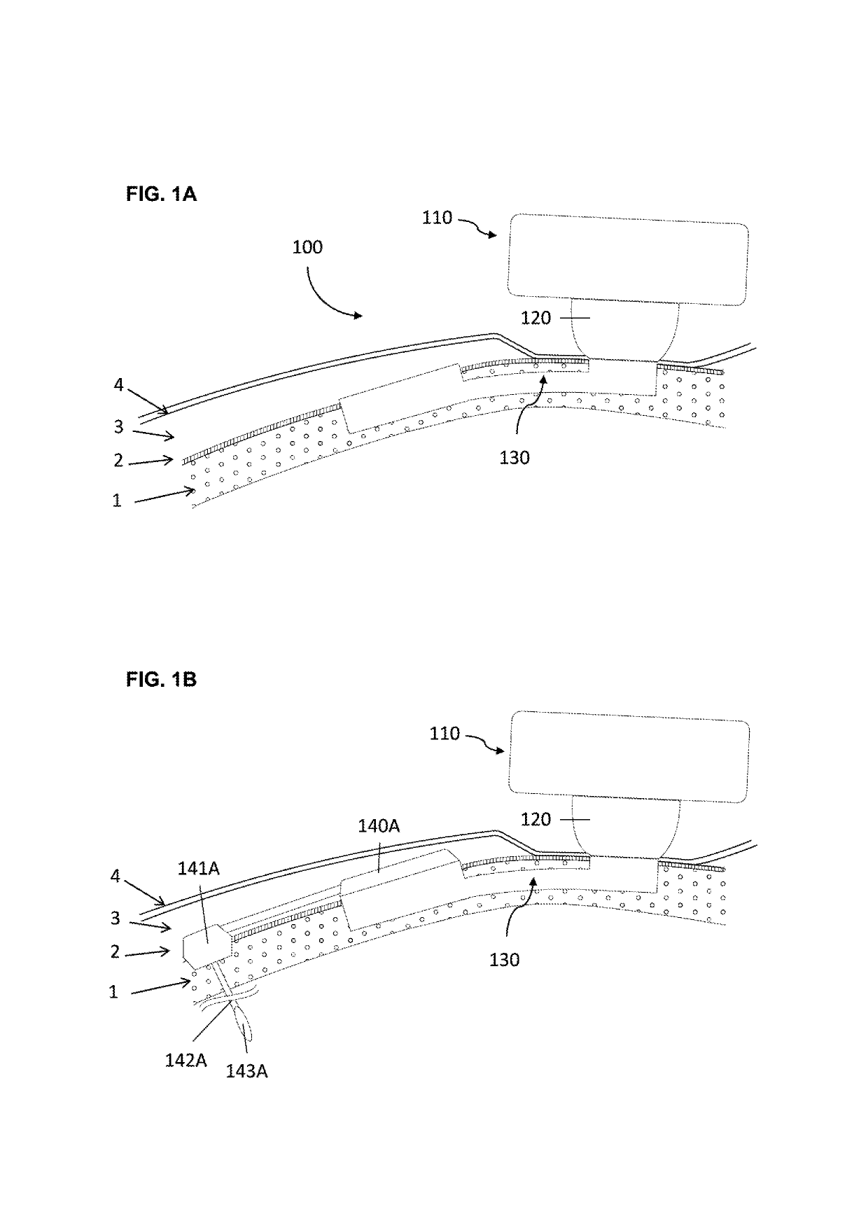

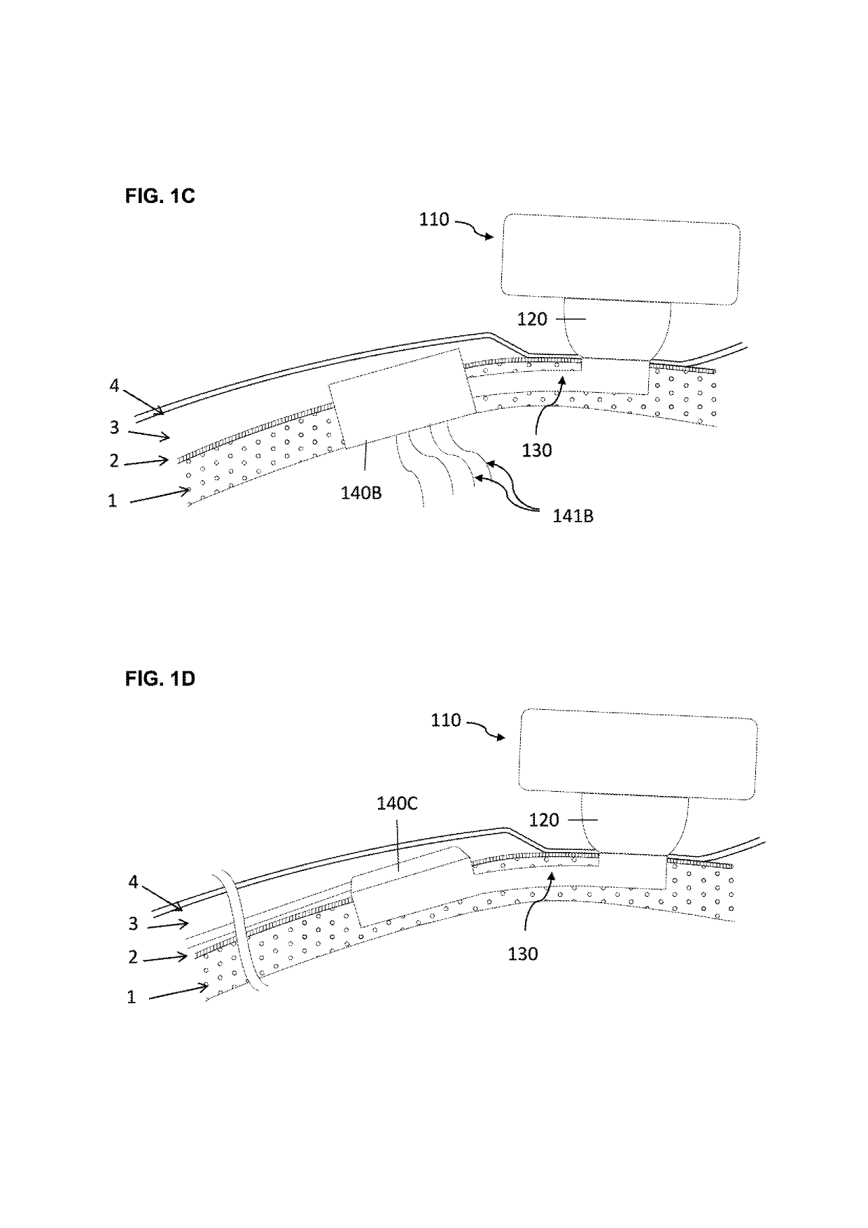

Considering the vast number of therapies for which the device can be used, several configurations can be set up. These configurations notably include:
the percutaneous system being associated with an intracerebral electrode (143A) maintained by a capsule (141A) and connected through a lead (142A) (FIG. 1B)
the percutaneous system being associated with a housing (140B) and associated electrodes (141B) such as described in the patent application published on Jun. 10, 2010 under the reference US 2010/0145162, which is incorporated by reference. (FIG. 1C)
the percutaneous system being associated with a cable (140C) that connects the external housing (110) to an internal entity. (FIG. 1D).) The cable can be a cervical cable in order to connect internal active entities located in caudal regions.

As is apparent from above, one arrangement consists in coupling the percutaneous connection device, i.e. the implant (130), with a connection housing specifically designed for enabling the connection elements to reach the other side of the osseous structure with regard to the side where the abutment (120) is positioned.

In particular, when the assembly is designed to be implanted in the cranium of a patient, such arrangement enables placing connectors, such as electrodes, facing the brain of a patient, for electrical stimulation for instance. Further, the specific configuration of the implant (130) enables shifting these connectors away from the percutaneous passage of the abutment (120), thus reducing the risks of propagation of a potential infection.

The arrangement illustrated in FIG. 1C comprises a connection housing (140B) which is directly connected to the extension member (130) forming the anchoring portion of the implant. As illustrated, such connection housing enables wires to be located on the other side of the osseous structure. The volume of the connection housing may also be sufficient to further enclose components, for example electronic components, such as an integrated circuit, processor, etc.

The arrangement illustrated in FIG. 1B is slightly different, wherein the connection housing is made of a proximal housing (140A) connected to the extension member (130) which is connected to a capsule (141B), said capsule enabling the passage through the osseous structure to the other side the structure. In the embodiment illustrated in FIG. 1B, the assembly comprises a single capsule (141A) having a single wire (142A) with an electrode (143A), but the assembly may include several capsules of this design, and/or the capsule can include several wires.

Most of the existing electrical active devices could be powered by the system described above.

In particular, the proposed percutaneous connection assembly is of interest in any of the following applications: hearing assistance, neuromodulation (deep brain stimulation, sacred nerve stimulation, spinal cord stimulation, vagus nerve stimulation, etc.), cardiac stimulations (ICD or CRT), artificial retina, mechanical circulatory support, etc. Alternatively the device described can be used for therapies using drug delivery and/or dialysis.

New therapies could also be investigated with the proposed active device.

First, new electrical therapies requiring a large amount of electricity can be develop. Several muscles could be stimulated through this system without batteries limitation. Exoskeleton could also interact with the data collected from the active medical device. Sphincter systems and artificial organs such as artificial kidney or artificial pancreas could be considered.

Second, the device can be used to transfer drugs, hormones to the human body or extract corporal liquids from the human body In new way.

Third, this device can be used for light therapies such as optogenetics therapies

Finally, this system can favour experimental work by letting surgeons and engineers access to the hardware and therefore replace components and reprogram the system easily.

1. A percutaneous connection assembly intended to be fixed by osseointegration in an osseous structure of a patient for transfer of a flow —preferably of electricity, light, data, or matter —between the interior and exterior of the body of the patient, wherein the assembly comprises:
An implant device having:
a percutaneous socket having a first end comprising a percutaneous abutment and a second end opposite to the first end;
an elongated extension member intended to be inserted within a hole created into the osseous structure, said extension member having a first end coupled to the second end of the socket, and a second end opposite to the first end, the coupling of the extension member relative to the percutaneous socket being designed for angular shifting of the first end of the percutaneous socket relative to the second end of the extension member; and
connection elements running through the device from the first end of the percutaneous socket to the second end of the extension member, said connection elements comprising at least a first connector arranged within the percutaneous abutment;
An external device for emission and/or reception of the flow toward and/or from the second end of the extension member, said external device including a housing with coupling elements for direct coupling of the housing onto the percutaneous abutment external to the body of the patient.

2. The percutaneous connection assembly of claim 1, wherein the external device is configured to convey an electrical signal, the housing of the external device containing one or several of elements among batteries, electronics, cables, connectors, circuitry, coils and any other elements for emission, reception, transfer, storage and/or processing of electrical signals.

3. The percutaneous connection assembly of claim 1, wherein the external device is configured to convey liquids and/or solids, the housing of the external device containing one or several of elements among pipes, tanks, valves, and/or pumps, and any other elements for emission, reception, transfer, storage and/or treatment liquids and/or solids.

4. The percutaneous connection assembly of claim 1, wherein the external device is configured to convey light, the external housing containing one or several of elements among laser and associated electronics, optical fibers, sensors and associated electronics, LED, mirrors, and any other optical elements for emission, reception, transfer, and/or storage of light.

5. A percutaneous connection device intended to be fixed by osseointegration in an osseous structure of a patient for transfer of a flow—preferably of electricity, light, data, or matter—between inside the body of the patient and an entity external to said body, wherein the device comprises:
a percutaneous socket having a first end comprising a percutaneous abutment and a second end opposite to the first end;
an elongated extension member intended to be inserted within a hole created into the osseous structure, said extension member having a first end coupled to the second end of the socket, and a second end opposite to the first end, the coupling of the extension member relative to the percutaneous socket being designed for angular shifting of the first end of the percutaneous socket relative to the second end of the extension member;
connection elements running through the device from the first end of the percutaneous socket to the second end of the extension member, said connection elements comprising at least a first connector arranged within the percutaneous abutment on one side of the osseous structure; and
a connection housing coupled at the second end of the elongated extension member, said connection housing being designed for bringing a second connector of the connection elements on the other side of the osseous structure.

6. The device of claim 5, wherein the connection housing is directly connected to the elongated extension member, said connection housing being designed to enclose several components therein.

7. The device of claim 5, wherein the connection housing comprises at least a capsule coupled to the elongated extension member with a flexible connecting member, said capsule having a design enabling a passage through the osseous structure.

8. The device of claim 5, comprising an external device for emission and/or reception of the flow toward and/or from the second end of the extension member, said external device including a housing with coupling elements for direct coupling of the housing onto the percutaneous abutment external to the body of the patient.
